(19) United States
(12) Patent Application Publication
(10) Pub. No.: US 2011/0280850 A1
(43) Pub. Date: Nov. 17, 2011

(54) Compositions Containing DNA Repair Enzyme And Anogeissus Extract

(76) Inventors: Elizabeth I. Starr, Great Neck, NY (US); Lieve Declercq, Ekeren (BE); Matthew Ronald Warren, South Huntington, NY (US); Christine Marie Baier, Massapequa Park, NY (US)

(21) Appl. No.: 12/778,726

(22) Filed: May 12, 2010

(51) Int. Cl. A61K 8/66; A61Q 19/08; A61Q 19/00

(52) U.S. Cl. 424/94.1

(57) ABSTRACT

A topical composition comprising at least one extract from the Anogeissus genus and at least one DNA repair enzyme and a method for treating human skin for improvement comprising applying to the skin a topical composition comprising at least one extract from the Anogeissus genus and at least one DNA repair enzyme.

### TECHNICAL FIELD

The invention is in the field of topical compositions for application to keratinous surfaces such as skin, hair, or nails containing at least one DNA repair enzyme and an Extract from the Anogeissus plant.

### BACKGROUND OF THE INVENTION

Companies that make skin care products are always looking for new and improved ingredients and formulas. It is known that skin cells, or keratinocytes, are vulnerable to daily assaults from the environment such as cigarette smoke, wind, sun, environmental toxins, and so on. The use of DNA repair enzymes in skin care products is known. It is believed that these enzymes are capable of improving the adverse effects of DNA damage in keratinocytes. Enhancing the beneficial effects of DNA repair enzymes by including other active ingredients in the skin care formulations is advantageous. It has been discovered that when DNA repair enzymes are combined with certain other ingredients including an extract from a plant in the Anogeissus genus, the result is a skin care product that provides improved skin texture, feel, hydration, moisturization, and appearance of lines, wrinkles, uneven pigmentation, mottling, and other age-related or undesirable skin conditions.

### SUMMARY OF THE INVENTION

The invention is directed to a topical composition comprising at least one extract from the Anogeissus genus and at least one DNA repair enzyme.

The invention is also directed to a method for treating human skin for improvement comprising applying to the skin a topical composition comprising at least one extract from the Anogeissus genus and at least one DNA repair enzyme.

### DETAILED DESCRIPTION

All percentages mentioned herein are percentages by weight unless otherwise indicated. The compositions of the invention may be further described as set forth herein.

### I. Anogeissus Extract

Anogeissus is a genus of trees that is indigenous to Asia and Africa, belonging to the family Combretaceae. There are about eight species in the Anogeissus genus, such as Anogeissus acumintata, bentii, dhofarica, latifolia, leiocarpus, rotundifolia, schimperi, and sericea. The extracts may come from the leaves, stems, seeds, bark, flowers, roots, and so on. In one embodiment the extract may be an aqueous or aqueous/alcoholic extract of the plant parts. The Anogeissus extract may be present in the composition in amounts ranging from about 0.0001-75%, preferably from about 0.0005-65%, more preferably about 0.001-50%.

Most preferred is an extract from the bark of Anogeissus Leiocarpus. One form is a brownish powder sold by Actives International LLC under the trade name ViaPure Anogeissus.

### II. DNA Repair Enzyme

The composition of the invention also contains at least one DNA repair enzyme. Suggested ranges are from about 0.00001 to about 35%, preferably from about 0.00005 to about 30%, more preferably from about 0.0001 to about 25% of one or more DNA repair enzymes.

DNA repair enzymes as disclosed in U.S. Pat. Nos. 5,077,211; 5,190,762; 5,272,079; and 5,296,231, all of which are hereby incorporated by reference in their entirety, are suitable for use in the compositions and method of the invention. One example of such a DNA repair enzyme may be purchased from AGI/Dermatics under the trade name Roxisomes®, and has the INCI name Arabidopsis Thaliana extract. It may be present alone or in admixture with lecithin and water. This DNA repair enzyme is known to be effective in repairing 8-oxo-diGuanine base mutation damage.

Another type of DNA repair enzyme that may be used is one that is known to be effective in repairing 06-methyl guanine base mutation damage. It is sold by AGI/Dermatics under the tradename Adasomes®, and has the INCI name Lactobacillus ferment, which may be added to the composition of the invention by itself or in admixture with lecithin and water.

Another type of DNA repair enzyme that may be used is one that is known to be effective in repairing T-T dimers. The enzymes are present in mixtures of biological or botanical materials. Examples of such ingredients are sold by AGI/Dermatics under the tradenames Ultrasomes® or Photosomes®. Ultrasomes® comprises a mixture of Micrococcus lysate (an end product of the controlled lysis of various species of Micrococcus), lecithin, and water. Photosomes® comprises a mixture of plankton extract (which is the extract of marine biomass which includes one or more of the following organisms: thalassoplankton, green micro-algae, diatoms, greenish-blue and nitrogen-fixing seaweed), water, and lecithin.

Another type of DNA repair enzyme may be a component of various inactivated bacterial lysates such as Bifida lysate or Bifida ferment lysate, the latter a lysate from Bifido bacteria which contains the metabolic products and cytoplasmic fractions when Bifido bacteria are cultured, inactivated and then disintegrated. This material has the INCI name Bifida Ferment Lysate.

Other suitable DNA repair enzymes include Endonuclease V, which may be produced by the denV gene of the bacteriophage T4. Also suitable are T4 endonuclease; O⁶-methylguanine-DNA methyltransferases; photolyases such as uracil- and hypoxanthine-DNA glycosylases; apyrimidinic/apurinic endonucleases; DNA exonucleases, damaged-bases glycosylases (e.g., 3-methyladenine-DNA glycosylase); correndonucleases either alone or in complexes (e.g., E. coli uvrA/uvrB/uvrC endonuclease complex); APEX nuclease, which is a multi-functional DNA repair enzyme often referred to as “APE”; dihydrofolate reductase; terminal transferase; topoisomerase; O⁶ benzyl guanine; DNA glycosylases.

Other types of suitable DNA repair enzymes may be categorized by the type of repair facilitated and include BER (base excision repair) or BER factor enzymes such as uracil-DNA glycosylase (UNG); single strand selective monofunctional uracil DNA glycosylase (SMUG1); 3,N(4)-ethenocytosine glycosylase (MBD4); thymine DNA-glycosylase (TDG); A/G-specific adenine DNA glycosylase (MUTYH); 8-oxoguanine DNA glycosylase (OGG1); endonuclease III-like (NTHL1); 3-methyladenine DNA glycosidase (MPG); DNA glycosylase/AP lyase (NEIL1 or 2); AP endonuclease (APEX 1 and 2), DNA ligase (LIG3), ligase accessory factor (XRCC1); DNA 5′-kinase/3′-phosphatase (PNKP); ADP-ribosyltransferase (PARP1 or 2).

Another category of DNA repair enzymes includes those that are believed to directly reverse damage such as O⁶-MeG alkyl transferase (MGMT); 1-meA dioxygenase (ALKBH2 or ALKBH3).

Yet another category of enzymes operable to repair DNA/protein crosslinks includes Tyr-DNA phosphodiesterase (TDP1).

Also suitable are MMR (mismatch excision repair) DNA repair enzymes such as MutS protein homolog (MSH2); mismatch repair protein (MSH3); mutS homolog 4 (MSH4); MutS homolog 5 (MSH5); or G/T mismatch-binding protein (MSH6); DNA mismatch repair protein (PMS1, PMS2, MLH1, MLH3); Postmeiotic segregation increased 2-like protein (PMS2L3); or postmeiotic segregation increased 2-like 4 pseudogene (PMS2L4).

Also suitable are DNA repair enzymes are those known as nucleotide excision repair (NER) enzymes and include those such as Xeroderma pigmentosum group C-complementing protein (XPC); RAD23 (S. cerevisiae) homolog (RAD23B); caltractin isoform (CETN2); RFA Protein 1, 2, of 3 (RPA1, 2, or 3); 3′ to 5′ DNA helicase (ERCC3); 5′ to 3′ DNA helicase (ERCC2); basic transcription factor (GTF2H1, GTF2H2, GTF2H3, GTF2H4, GTF2H5); CDK activating kinase (CDK7, CCNH); cyclin G1-interacting protein (MNAT1); DNA excision repair protein ERCC-51; excision repair cross-complementing 1 (ERCC1); DNA ligase 1 (LIG1); ATP-dependent helicase (ERCC6); and the like.

Also suitable may be DNA repair enzymes in the category that facilitate homologous recombination and include, but are not limited to DNA repair protein RAD51 homolog (RAD51, RAD51L1, RAD51B etc.); DNA repair protein XRCC2; DNA repair protein XRCC3; DNA repair protein RAD52; ATPase (RAD50); 3′ exonuclease (MRE11A); and so on.

DNA repair enzymes that are DNA polymerases are also suitable and include DNA polymerase beta subunit (POLB); DNA polymerase gamma (POLG); DNA polymerase subunit delta (POLD1); DNA polymerase II subunit A (POLE); DNA polymerase delta auxiliary protein (PCNA); DNA polymerase zeta (POLZ); MAD2 homolog ((REV7); DNA polymerase eta (POLH): DNA polymerase kappa (POLK): and the like.

Various types of DNA repair enzymes that are often referred to as “editing and processing nucleases” include 3′-nuclease; 3′-exonuclease; 5′-exonuclease; endonuclease; and the like.

Other examples of DNA repair enzymes include DNA helicases including such as ATP DNA helicase and so on.

The DNA repair enzymes may be present as components of botanical extracts, bacterial lysates, biological materials, and the like. For example, botanical extracts processed in a certain manner may contain DNA repair enzymes.

The compositions of the invention may contain one or more DNA repair enzymes. Most preferred are NDA repair enzymes OGGI, OGGI fermentate, Micrococcus luteus, and mixtures thereof or ferments thereof, and in the amounts set forth herein.

### III. Other Ingredients

The composition may be in the form of an aqueous solution, gel, or suspension; or in the form of an emulsion—either water in oil or oil in water. The composition may also be anhydrous. The composition may be in the liquid, semi-solid, or solid form.

If present as an aqueous solution or dispersion, the amount of water present may range from about 0.01-99%, and the amount of dissolved or dispersed solids from about 10 to 99.99%, in addition to the DNA repair enzyme and Anogeissus extract mentioned about in the amounts set forth.

If the composition of the invention is in the emulsion form, it may comprise from about 0.1-99% water and from about 0.1-80% oil in addition to DNA repair enzyme and Anogeissus extract as specified herein and in the amounts set forth.

If the composition of the invention is in an anhydrous form, it may contain from about 0.1-99% oil in addition to the Anogeissus extract and DNA repair enzymes and in the amounts set forth herein.

A. Humectants

The composition may contain one or more humectants. If present, they may range from about 0.1 to 75%, preferably from about 0.5 to 70%, more preferably from about 0.5 to 40%. Examples of suitable humectants include glycols, sugars, and the like. Suitable glycols are in monomeric or polymeric form and include polyethylene and polypropylene glycols such as PEG 4-10, which are polyethylene glycols having from 4 to 10 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol, glycerin, propylene glycol, or hexylene glycol.

B. Botanical Extracts

It may be desirable to incorporate one more botanical extracts into the composition in addition to the Anogeissus extract. If present suggested ranges are from about 0.0001 to 20%, preferably from about 0.0005 to 15%, more preferably from about 0.001 to 10%. Suitable botanical extracts include extracts from plants (herbs, roots, flowers, fruits, seeds) such as flowers, fruits, vegetables, and so on, including yeast ferment extract, Padina Pavonica extract, Thermus Thermophilis ferment extract, Camelina Sativa seed oil, Boswellia Serrata extract, olive extract, Acacia Dealbata extract, Acer Saccharinum (sugar maple), Acidopholus, Acorus, Aesculus, Agaricus, Agave, Agrimonia, algae, aloe, citrus, Brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Prunus Amygdalus, Rosmarinus Officianalis, Sapindus makurossi, Caesalpinia spinosa, Citrus Medica Limonum, Panax Ginseng, Siegesbeckia Orientalis, Mangifera Indicia, Fructus Mume, Psidium Guajava, Ascophyllum Nodosum, Centaurium erythrea, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, Kola Acuminata, glycyrretinic acid, and mixtures thereof.

C. Peptides

It may be desired to incorporate one or more peptides into the composition. The term “peptide” means from 2 to 20 amino acids connected by peptide bonds. If so, suggested ranges are from about 0.001 to 20%, preferably from about 0.005 to 15%, more preferably from about 0.01 to 10%. Preferred are biologically active peptides including those set forth in the C.T.F.A. International Cosmetic Ingredient Dictionary and Handbook, Eleventh Edition, 2006, page 2712, hereby incorporated by reference its entirety. Such peptides include, but are not limited to the CTFA names: Acetyl Hexapeptide-1, 7, 8; Acetyl Pentapeptide-1, 2, 3, or 5; Acetyl Tripeptide-1; Acetyl Dipeptide-1 cetyl ester; Acetyl Glutamyl Heptapeptide-3; Acetyl Glutamyl Hexapeptide-6; Acetyl Monofluoropeptide-1; Heptapeptide-1, 2, or 3; Hexapeptide-1, 2, 3, 4, 5, 6, 7, 8, 9, 10, 11, 12, 13, or 14; Manganese Tripeptide-1; Myristoyl Hexapeptide-5, 12, or 13; Myristoyl Nonapeptide-2; Myristoyl Pentapeptide-4; Myristoyl Tetrapeptide-4 or 6; Myristoyl Tripeptide-4; Nisin, Nonapeptide-1 or 2; Oligopeptide-1, 2, 3, 4, 5, 6, 7, 8, 9 or 10; Palmitoyl Hexapeptide-14; Palmitoyl Petnapeptide-4; Palmitoyl Pentapeptide-4 or 5; Palmitoyl Tripeptide-1 or 5; Pentapeptide-1, 2, 3, 4, 5, or 6; Tetrapeptide-1, 2, 3, 4, 5, 6, or 7; Tripeptide-1, 2, 3, 4, or 5.

In one preferred embodiment the composition comprises Acetyl Hexapeptide-8, having the trade name Argireline®.

D. Oils

The composition may also comprise one or more oils in the form of natural, synthetic, or silicone oils. The term “oil” means that the ingredient is pourable at room temperature, e.g. 25° C. Oils may be volatile or non-volatile. The term “volatile” means that the oil has vapor pressure greater than about 2 mm of mercury at 20° C. The term “non-volatile” means that the oil has a vapor pressure of less than about 2 mm. of mercury at 20° C. If present, suggested ranges are from about 0.1 to 60%, preferably from about 0.5 to 45%.

Examples of volatile oils include volatile linear, cyclic or branched silicones such as cyclopentasiloxane, cyclohexasiloxane (2 cst), hexamethyldisiloxane (0.65 cst, centistokes), octamethyltrisiloxane (1.0 cst), decamethyltetrasiloxane (1.5 cst), or dodecamethylpentasiloxane (2.0 cst); or branched volatile silicones such as methyl trimethicone (1.5 cst). Also suitable are volatile paraffinic hydrocarbons such as isododecane, isohexadecane, C11-14 alkanes, and mixtures thereof.

Non-volatile oils include linear silicones commonly referred to as dimethicone; phenyl substituted silicones such as phenyl dimethicone, phenyl trimethicone, trimethylsiloxy phenyldimethicone, cetyl dimethicone, perfluorodimethicone, phenethyl dimethicone, and the like.

Non-volatile oils may also include esters or hydrocarbons. Esters include C1-10 alkyl esters of C1-20 carboxylic acids. One preferred type of ester is a fatty acid (C6-22) ester of a straight or branched chain saturated or unsaturated C1-22 alkyl. Examples include esters that have a low viscosity, e.g. ranging from 10-100 cst at room temperature. Examples of such esters include but are not limited to jojoba esters.

Other non-volatile oils include sterols such as phytosterols, phytosphingosine, and similar plant sterols.

E. Thickeners

Suitable thickeners may be incorporated into the composition. If so, suggested ranges are from about 0.0001-45%, preferably from about 0.0005-40%.

Examples of thickeners include animal, vegetable, mineral, silicone, or synthetic waxes which may have melting points ranging from about 30 to 150° C. including but not limited to Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.

Also suitable as thickening agents are silicas, silicates, silica silylate, and alkali metal or alkaline earth metal derivatives thereof. These silicas and silicates are generally found in the particulate form and include silica, silica silylate, magnesium aluminum silicate, and the like.

Silicone elastomers may also be used as thickening agents. Such elastomers include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or a network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo K K, each of which are herein incorporated by reference in its entirety.

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are different types of synthetic polymeric thickeners. One type includes acrylic polymeric thickeners comprised of monomers A and B wherein A is selected from the group consisting of acrylic acid, methacrylic acid, and mixtures thereof; and B is selected from the group consisting of a C₁₋₂₂ alkyl acrylate, a C₁₋₂₂ alky methacrylate, and mixtures thereof are suitable. Acrylic polymer solutions include those sold by Seppic, Inc., under the tradename Sepigel® or those sold under the tradename Aristoflex®.

Also suitable are acrylic polymeric thickeners that are copolymer of A, B, and C monomers wherein A and B are as defined above, and C has the general formula:

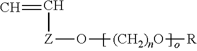

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid or mixtures thereof, and where the allyl ether unit containing a fatty chain corresponds to the monomer of formula:

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl (such as 6-40, 10-30, and the like) groups, for example acrylates/C₁₀₋₃₀ alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.

Also suitable as thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CTFA names polyglycerin-20, polyglycerin-40, and the like.

F. Surfactants

If desired, the compositions of the invention may contain one or more surfactants. This is particularly desirable when the composition is in the form of an aqueous gel or emulsion. If present, the surfactant may range from about 0.001 to 50%, preferably from about 0.005 to 40%, more preferably from about 0.01 to 35% by weight of the total composition. Suitable surfactants may be silicone or organic, nonionic, anionic, amphoteric or zwitterionic. Such surfactants include, but are not limited to, those set forth herein.

Suitable silicone surfactants include polyorganosiloxane polymers that have amphiphilic properties, for example contain hydrophilic radicals and lipophilic radicals. These silicone surfactants may be liquids or solids at room temperature.

One type of silicone surfactant that may be used is generically referred to as dimethicone copolyol or alkyl dimethicone copolyol. It may be either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

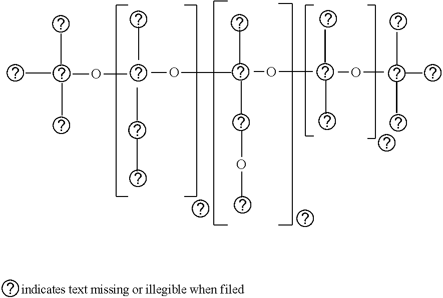

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Crosslinked silicone surfactants, often referred to as emulsifying elastomers are suitable. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference. Polyoxyalkylenated silicone elastomers that may be used include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One example of a crosslinked silicone elastomer emulsifier that may be used is dimethicone/PEG-10/15 crosspolymer, which provides excellent aesthetics due to its elastomeric backbone, but also surfactancy properties.

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, Laureth 2-100, formed by the reaction of lauryl alcohol and ethylene oxide where the number of repeating ethylene oxide units is 2 to 100, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000. Also suitable are ethoxylated propoxylated derivatives of C6-30 saturated or unsaturated fatty acids, for example, Di-PPG-2 myreth-10 adipate, Di-PPG-2 Ceteth-4 adipate, Di-PPG Myristyl Ether Adipate,

Other nonionic surfactants that may be used are formed by the reaction of a carboxylic acid with an alkylene oxide or with a polymeric ether or monomeric, homopolymeric, or block copolymeric ethers; or alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C6-30, preferably C12-22 fatty acids. Examples of such ingredients include Polysorbates 20-85, sorbitan oleate, sorbitan sesquioleate, sorbitan palmitate, sorbitan sesquiisostearate, sorbitan stearate, and so on.

G. Vitamins and Antioxidants

It may be desirable to incorporate one or more vitamins or antioxidants in the compositions. If present, suggested ranges are from about 0.001 to 20%, preferably from about 0.005 to 15%, more preferably from about 0.010 to 10%. Preferably such vitamins, vitamin derivatives and/or antioxidants are operable to scavenge free radicals in the form of singlet oxygen. Such vitamins may include tocopherol or its derivatives such as tocopherol acetate, tocopherol ferulate; ascorbic acid or its derivatives such as ascorbyl palmitate, magnesium ascorbyl phosphate; Vitamin A or its derivatives such as retinyl palmitate; or vitamins D, K, B, or derivatives thereof.

The invention will be further described in connection with the following example which is set forth for purposes of illustration only.

### Example 1

A skin treatment serum is prepared as follows:

| Ingredient | Wt % |
|---|---|
| Water | QS100 |
| Butylene glycol | 5.20 |
| Dimethicone | 4.25 |
| Acetyl hexapeptide-8 | 2.00 |
| Arginine/salicylic acid/tocopheryl acetate/mixed | 2.00 |
| soy phospholipids | |
| Jojoba esters | 1.50 |
| Glycerin | 1.25 |
| Dicaprylyl carbonate | 1.00 |
| Methyl trimethicone | 1.00 |
| Dimethicone/Polysilicone-11 | 1.00 |
| Water/Prunus amygdalus ducis (sweet almond) seed extract | 1.00 |
| Silica | 0.75 |
| Carbomer | 0.50 |
| Ammonium acryloyldimethyltaurate/VP copolymer | 0.50 |
| Glycerin/water/Sapindus mukurossi fruit | 0.50 |
| extract/Caesalpinia spinosa gum | |
| Butylene glycol/Centaurium erythraea (Centaury) extract | 0.50 |
| Siegesbeckia Orientalis extract/glycerin | 0.50 |
| Tromethamine | 0.45 |
| Phenoxyethanol | 0.43 |
| Caprylyl glycol/phenoxyethanol/hexylene glycol | 0.40 |
| Caffeine | 0.20 |
| Water/butylenes glycol/Scutellaria baicalensis (root) | 0.20 |
| extract/Pyrus Malus | |
| (Apple) extract/Cucumis Sativus (cucumber) extract | |
| Tetrahexyldecyl ascorbate | 0.20 |
| Phytosphingosine | 0.20 |
| Anogeissus Leiocarpus extract | 0.15 |
| Fragrance | 0.15 |
| Glycyrretinic acid | 0.10 |
| Mangifera Indicia (Mango) leaf extract | 0.10 |
| Psidium Guajava (Guava) fruit extract | 0.10 |
| Zinc PCA | 0.10 |
| Polygonum cuspidatum root extract | 0.05 |
| Sodium hyaluronate | 0.02 |
| Phosphatidyl choline | 0.009 |
| Sodium chloride | 0.009 |
| Phytosterol | 0.0019 |
| Disodium phosphate | 0.001 |
| Ursolic acid/Rosmarinus Officinalis (Rosemary) extract | 0.001 |
| Anthemis Nobilis (Chamomile) flower extract | 0.0005 |
| Micrococcus Luteus powder | 0.0005 |
| Potassium phosphate monobasic | 0.0002 |
| Potassium chloride | 0.0002 |
| Phosphatidyl ethanolamine | 0.00009 |
| Disodium EDTA | 0.00006 |
| BHT | 0.00002 |
| Oleic acid | 0.00002 |
| Sodium hydroxide | 0.000007 |
| OGGI fermentate | 0.000002 |

The composition was prepared by combining the ingredients and mixing well.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A topical composition comprising at least one extract from the Anogeissus genus and at least one DNA repair enzyme.

2. The composition of claim 1 wherein the extract is from Anogeissus Leiocarpus.

3. The composition of claim 1 further comprising at least one peptide.

4. The composition of claim 1 comprising about 0.0001-75% of at least one extract from Anogeissus Leiocarpus; about 0.00001 to about 35% of at least one DNA repair enzyme; and about 0.001-20% of at least one peptide having from about 2 to 20 amino acids in an amount ranging from about 0.001 to 20%.

5. The composition of claim 4 wherein the DNA repair enzyme is operable to repair T-T dimer, 8-oxo-diGuanine, or 06-methyl guanine nucleotide base mutation damage.

6. The composition of claim 4 wherein the DNA repair enzyme is one or more of OGGI, or found in Micrococcus lysate, Bifidus, Arabidopsis Thaliana extract, Lactobacillus, plankton extract, or mixtures thereof, or fermentation products thereof.

7. The composition of claim 4 wherein the peptide is selected from a hexapeptide, pentapeptide, tripeptide, dipeptide or mixtures thereof.

8. The composition of claim 4 wherein the peptide is Acetyl Hexapeptide; Palmitoyl Hexapeptide, Palmitoyl Pentapeptide, Palmitoyl Tripeptide; Pentapeptide; Tetrapeptide; Tripeptide.

9. The composition of claim 4 wherein the DNA repair enzyme is one or more of OGGI, or found in Micrococcus lysate, Bifidus, Arabidopsis Thaliana extract, Lactobacillus, plankton extract, or mixtures thereof or ferments thereof; and the peptide comprises Acetyl Hexapeptide-8.

10. The composition of claim 9 further comprising caffeine.

11. The composition of claim 10 further comprising one or more of the botanical extracts from Scutellaria baicalensis; Pyrus malus; Cucumis sativus; glycyrretinic acid; Mangifera indicia; Psidium guava; Polygonum cuspidatum; Rosmarinus officinalis; Anthemis nobilis.

12. The composition of claim 11 further comprising a humectant selected from glycerin, butylene glycol, propylene glycol, or mixtures thereof.

13. The composition of claim 12 further comprising at least one silicone elastomer.

14. An aqueous based topical skin care composition comprising, by weight of the total composition:
about 0.0001-75% of at least one extract from Anogeissus Leiocarpus;
about 0.00001 to about 35% of at least one DNA repair enzyme comprising one or more of OGGI, or a DNA repair enzyme found in Micrococcus lysate, Bifidus, Arabidopsis Thaliana extract, Lactobacillus, plankton extract, or mixtures thereof or fermentation products thereof;
about 0.001-20% of at least one peptide selected from Acetyl hexapeptide-8; Palmitoyl oligopeptide or mixtures thereof;
about 0.1-60% of a an oil selected from methyl trimethicone, dimethicone, and mixtures thereof;
about 0.0001-25% of a botanical extract selected from the group consisting of Scutellaria baicalensis; Pyrus malus; Cucumis sativus; glycyrretinic acid; Mangifera indicia; Psidium guava; Polygonum cuspidatum; Rosmarinus officinalis; Anthemis nobilis and mixtures thereof; and
about 0.1 to 75% of a humectant selected from glycerin, propylene glycol, butylene glycol or mixtures thereof.

15. The composition of claim 14 additionally comprising about at least one thickening agent comprising ammonium acryloyldimethyltaurate/VP copolymer; ammonium acryloyldimethyltaurate/beheneth-25 methacrylate crosspolymer, or mixtures thereof.

16. The composition of claim 15 additionally comprising at least one thickening agent which is a silicone elastomer.

17. The composition of claim 1 further comprising a thickening agent that is a silicone elastomer.

18. The composition of claim 1 further comprising a thickening agent selected from ammonium acryloyldimethyltaurate/VP copolymer; ammonium acryloyldimethyltaurate/beheneth-25 methacrylate crosspolymer, or mixtures thereof.

19. The composition of claim 17 wherein the silicone elastomer comprises Polysilicone-11.

20. The composition of claim 1 wherein the thickening agent comprises a mixture of Polysilicone-11 and a acryloyldimethyltaurate polymer.
